SEQUENCE LISTING (4 sequences)

SEQ ID NO: 1 - protein, 102 aa, Artificial Sequence
  VARIANT (2)..(2): Xaa can be any amino acid
LXDXLXXLXXXXXXXXXXXXXXXXXXXXXXXXXXXXXXXXXXXXXXXXXXXXXXXXXXXXXXXXXXXXXXXXXXXXXXXXXXXXXXXXLXCXEXXXXXSDDE

SEQ ID NO: 2 - DNA, 26 bp, Homo sapiens
ggaagcagaccacgtggtctgcttcc

SEQ ID NO: 3 - protein, 7 aa, Simian virus 40
PKKKRKV

SEQ ID NO: 4 - DNA, 16 bp, Homo sapiens
cggacaatgttgaccg

(19) United States
(12) Patent Application Publication
(10) Pub. No.: US 2002/0098166 A1
(43) Pub. Date: Jul. 25, 2002

(54) GENETICALLY MODIFIED CELLS AND THEIR USE IN THE PROPHYLAXIS OR THERAPY OF DISORDERS

(76) Inventors: KLAUS HAVEMANN, MARBURG (DE); DR.ROLF MUELLER, MARBURG (DE); DR.HANS-HARALD SEDLACEK, MARBURG (DE)

Correspondence Address: Patricia D. Granados, Heller Ehrman White & McAuliffe LLP, 1666 K Street, NW, Suite 300, WASHINGTON, DC, 20006-1228, US

(21) Appl. No.: 09/119,659

(22) Filed: Jul. 21, 1998

(30) Foreign Application Priority Data
Jul. 21, 1997 (DE) 19731154.7
Nov. 26, 1997 (DE) 19752299.8

(51) Int. Cl. A61K 48/00; C12N 5/06

(52) U.S. Cl. 424/93.1; 435/325

(57) ABSTRACT

The invention provides a method of culturing mononuclear cells, comprising isolating mononuclear cells from the blood or cell-containing fluids of the body of a mammal; culturing the cells in a culture medium comprising one or more of gangliosides, phospholipids, glycolipids and growth factors for endothelial cells. These cells are useful, for example, for the endothelialization of injured vessels. The invention also provides a method of making cells capable of expressing a biologically active protein, comprising isolating mononuclear cells from the blood or cell-containing fluids of the body of a mammal; culturing the cells in a culture medium comprising one or more of gangliosides, phospholipids, glycolipids and growth factors for endothelial cells; optionally, before or after culturing the cells, immortalizing the cells; and transfecting the cells with a nucleic acid construct comprising a gene for the biologically active protein. The cells are useful, for example in gene therapy methods for the prophylaxis or therapy of disorders. Cells obtainable by these methods and obtained by these methods also are provided.

### BACKGROUND OF THE INVENTION

The administration of somatic cells transfected or transduced in vitro is a gene therapy method which is presently widely used in testing preclinically and clinically. Different cells, including fibroblasts, lymphocytes, keratinocytes and tumor cells, have been transduced to express an active compound.

Endothelial cells were used for this purpose for the first time in 1989. To this end, endothelial cells were transfected in vitro with the aid of a retroviral vector to express an active compound. Zwiebel et al., Science 243: 220 (1989). Transduced endothelial cells of this type were grown in vitro on plastic blood vessel prostheses. After in vivo transplantation of these protheses, the transduced cells were able to express the transgene. Id.; Wilson et al., Science 244: 1344 (1989). Zwiebel and Wilson proposed administering transduced endothelial cells adhered to a plastic or collagen support to patients for the purposes of gene therapy. This proposal was carried out experimentally by Nathan et al., P.N.A.S., USA 92: 8130 (1995).

The ability to effect gene therapy by administering a cell suspension comprising transduced endothelial cells into the blood stream was shown for the first time by Nabel et al., Science 244: 1342 (1989). The authors were able to show that endothelial cells obtained from vessels of a live mammal by scraping and transduced in vitro to express a reporter gene, after local administration, for example, to blood vessels having endothelial cell damage, grow there and express the reporter gene. On the basis of these results, the authors describe the possibility of administering genetically modified endothelial cells for the purposes of gene therapy, such that active compounds are delivered by the endothelial cells directly into blood circulation for the purposes of the therapy of systemic or hereditary disease. This idea was further developed by Bernstein et al., FASEB J. 4: 2665 (1990), which reports that pulmonary endothelial cells transfected with plasmids in vitro to express an active compound were administered to nude mice by injection intraperitoneally (i.p.), intravenously (i.v.), subcutaneously (s.c.) or under the renal capsule. The active compound produced by the endothelial cell transplants was detected locally (in cysts of the kidneys) or in the blood (after i.p., i.v. or s.c. administration) of treated animals, demonstrating the utility of the in vivo administration of endothelial cells transduced in vitro for gene therapy.

Zwiebel et al., WO93/13807 (1992) and Ojeifo et al., Cancer Res. 55: 2240 (1995) showed in a number of examples the possibility of using endothelial cells transduced in vitro for gene therapy by introducing them into blood circulation. WO93/13807 reports that human umbilical cord endothelial cells and endothelial cells from the fatty tissue of rats were transduced in vitro using a retroviral vector to express an active compound and injected intravenously into animals in which local vascular damage and angiogenesis had first been produced by the injection of irradiated FGF-secreting cells. The injected endothelial cells are reported to have localized at the site of the vascular damage and angiogenesis and to have expressed the active compound there. Against this background, the authors claimed in their patent application the use of in vitro transduced endothelial cells for the expression of adenosine aminase, blood clotting factors, hematopoietic growth factors, cytokines, antithrombotics, enzyme inhibitors and hormones.

In parallel to these studies, techniques for isolating endothelial cells were improved, and the migration behavior of endothelial cells transduced in vitro was studied in greater detail by other authors. Messina et al., P.N.A.S., USA 89: 12018 (1992), were able to show that endothelial cells transfected in vitro and injected into blood circulation can adhere to and integrate into intact endothelial cells. This implied that intravascularly administered endothelial cells do not localize exclusively in zones with vascular damage and angiogenesis. On the other hand, it has been shown that endothelial cells transfected in vitro and injected as a mixture with tumor cells are involved in the angiogenesis of the tumor vascular bed. Lal et al., P.N.A.S., USA 91: 9695 (1994); Nam et al., Brain Res. 731: 161 (1996). Owing to this, tumor cells transplanted as a mixture with endothelial cells have a distinct growth advantage in vivo. Stopeck et al., Proc. Am. Assoc. Cancer Res. 38: 265 (1997).

Transduced endothelial cells can be pharmacologically active or have antitumor activity locally, e.g., when administered into the brain or into a brain tumor, by expression of the active compound encoded by the transgene. Nam et al., Brain Res. 731: 161 (1996); Quinonero et al., Gene Ther. 4: 111 (1997). Robertson et al., Proc. Am. Assoc. Cancer Res. 38: 382 (1997), used human endothelial cells (HUVEC) that had been transduced in vitro using an AV vector to express HSV-TK. The cells were administered to nude mice as a mixture with human ovarian carcinoma cells. After administration of ganciclovir, which is activated in the tumor by the HSV-TK to give a cytostatic, marked tumor regression was observed in treated mice.

The use of endothelial cells as cellular carriers of transgenes in gene therapy has until now been considerably restricted by two significant problem areas:

(1) The ability to obtain suitable endothelial cells in sufficient number has until now proved to be extremely difficult.

Allogenic endothelial cells are relatively simple to obtain from the umbilical cord or from cell cultures, but as a result of their immunogenicity they can only be used in the recipient in a restricted manner. Moreover, their proliferation in cell culture is only possible to a restricted extent.

Autologous endothelial cells can be obtained, for example, mechanically by the scraping out of varicose veins or from fatty tissue. This type of procedure is not possible for all patients, and involves considerable injury to the patient. As an alternative, angioblasts or precursor cells of endothelial cells have been obtained from peripheral blood. Asahara et al., Science 275: 964 (1997). The collection of blood necessary for this is less stressful for patients; however, the isolation of the supposed angioblasts from mononuclear blood cells and the differentiation of these angioblasts into endothelial cells is very complicated. In a typical method, mononuclear CD34⁺ or Flk-1 positive blood cells which are present in the blood in only a low concentration (less than or equal to about 0.1%) are isolated from blood leukocytes (isolated, for example, with the aid of density gradient centrifugation) by immunoadsorption on carrier-bound monoclonal antibodies (for example, monoclonal antibodies specific for CD34 or Flk-1). Subsequently, these cells are layered in tissue dishes with collagen type 1 or fibronectin for approximately 4 weeks in bovine brain-containing culture medium for differentiation into endothelial cells and for proliferation. The proliferation of these cells is only possible to a restricted extent. In addition, the incubation of the endothelial cells with cerebral matter, e.g., with bovine brain, raises considerable safety problems.

(2) The migration of the endothelial cells and the selective expression of the transgene in the desired target area is not sufficiently controllable.

After intravascular administration of the endothelial cells, the cells localize both in regions of angiogenesis and on and in the resting endothelial cell layer, as described above. It is unclear whether endothelial cells which are formed in cell culture from precursor cells can redifferentiate into precursor cells again in vivo after injection and disperse over the entire body.

There is a need therefore, for improved methods of isolating, culturing and transfecting endothelial cells and endothelial precursor cells, and of targeting such cells to specific sites in vivo.

### SUMMARY OF THE INVENTION

It is one object of the present invention to provide methods of isolating endothelial cells or endothelial precursor cells. It is another object of the present invention to provide methods of culturing endothelial cells or endothelial precursor cells. It is another object of the present invention to provide methods of transfecting endothelial cells or endothelial precursor cells such that the cells are capable of expressing one or more biologically active proteins. It is another object of the invention to provide cells obtainable by these methods. It is another object of the present invention to provide methods of gene therapy. It is another object of the invention to provide methods of endothelializing injured vessels.

In accordance with these and other objects, the present invention provides, in accordance with one embodiment, a method of culturing mononuclear cells comprising isolating mononuclear cells from the blood or cell-containing fluids of the body of a mammal; culturing the cells in a culture medium comprising one or more of gangliosides, phospholipids, glycolipids and growth factors for endothelial cells, including those growth factors which influence differentiation, survival, migration and vascularization. In one particular embodiment, the culture medium comprises one or more growth factors for endothelial cells.

In accordance with another embodiment, the invention provides a method of making cells capable of expressing a biologically active protein comprising isolating mononuclear cells from the blood or cell-containing fluids of the body of a mammal; culturing the cells in a culture medium comprising one or more of gangliosides, phospholipids, glycolipids and growth factors for endothelial cells, including those growth factors which influence differentiation, survival, migration and vascularization; optionally, before or after culturing the cells, immortalizing the cells; and transfecting the cells with a nucleic acid construct comprising a gene for the biologically active protein. In one particular embodiment, the culture medium comprises one or more growth factors for endothelial cells. In one particular embodiment, the cells are immortalized by a process selected from the group consisting of transforming the cells with an exogenous oncogene; activating an endogenous oncogene; and inactivating an endogenous suppressor gene. In another particular embodiment, the nucleic acid construct comprises a promoter operably linked to the gene for the biologically active protein. In another particular embodiment, the nucleic acid construct comprises at least two promoters operably linked to the gene for the biologically active protein, which promoters may be the same or different.

In accordance with another embodiment, the invention provides a method of effecting gene therapy of a disorder comprising administering to a patient in need thereof a therapeutically effective amount of cells obtained by isolating mononuclear cells from the blood or cell-containing fluids of the body of a mammal; culturing the cells in a culture medium comprising one or more of gangliosides, phospholipids, glycolipids and growth factors for endothelial cells, including those growth factors which influence differentiation, survival, migration and vascularization; optionally, before or after culturing the cells, immortalizing the cells; and transfecting the cells with a nucleic acid construct comprising a gene for a biologically active protein useful in the prophylaxis or therapy of the disorder.

In accordance with another embodiment, the invention provides a method of endothelializing injured vessels comprising, administering to a patient in need thereof a therapeutically effective amount of cells obtained by isolating mononuclear cells from the blood or cell-containing fluids of the body of a mammal; culturing the cells in a culture medium comprising one or more of gangliosides, phospholipids, glycolipids and growth factors for endothelial cells; and optionally, before or after culturing the cells, immortalizing the cells.

In accordance with another embodiment, the invention provides a cell obtainable by (a) isolating mononuclear cells from the blood or cell-containing fluids of the body of a mammal; and (b) culturing the mononuclear cells in a cell culture medium comprising one or more of gangliosides, phospholipids, glycolipids and growth factors for endothelial cells.

In accordance with another embodiment, the invention provides a cell for use in gene therapy, obtainable by (a) isolating mononuclear cells from the blood or cell-containing fluids of the body of a mammal; (b) culturing the mononuclear cells in a cell culture medium comprising one or more of gangliosides, phospholipids, glycolipids and growth factors for endothelial cells; (c) optionally, before or after culturing the cells, immortalizing the cells by a process selected from the group consisting of transforming the cells with an exogenous oncogene; activating an endogenous oncogene; and inactivating an endogenous suppressor gene; and (d) transfecting the cells with a nucleic acid construct comprising a gene coding for a biologically active protein, wherein the nucleic acid construct optionally comprises one or more promoters for expressing the gene for the biologically active protein cell-specifically, cell cycle-specifically, virus-specifically, metabolically or by hypoxia. Pharmaceutical compositions comprising the cells also are provided.

These and other objects and advantages of the invention are described in the description that follows:

### DETAILED DESCRIPTION OF THE INVENTION

With the present invention, the two significant problem areas discussed above are now solved. In solving these problems, the invention provides:

(1) An improved, simple and safe method for isolating and culturing mononuclear cells, in particular, endothelial precursor cells, from the blood and other cell-containing fluids of the body of a mammal, and the use of these cells in gene therapy methods for the prophylaxis or therapy of disorders.

(2) A cell-specific, in particular, endothelial cell-specific, optionally pharmacologically controllable, transformation of these cells, such that, with the aid of cell culture, slightly greater amounts of such cells can be obtained.

(3) The production of cells, in particular endothelial cells, as vectors for effector genes, i.e., genes which code for biologically active compounds for the prophylaxis or therapy of a disorder. For example, at least one effector gene can be inserted into a cell, for example, an endothelial cell prepared as described in (1) or (1) and (2) above. The effector gene is expressed cell-specifically, in particular, endothelial cell-specifically, and, optionally, as a result of hypoxia, cell cycle-specifically, and/or virus-specifically by the selection of suitable promoter systems.

(4) The administration of these genetically modified cells, in particular, the endothelial cells obtained and modified as described above, for use in gene therapy for the prophylaxis or therapy of a disorder.

(5) The use of the cultured cells in in vitro pharmacological studies. For example, the cells can be used to search for and test compounds that influence the growth or function of endothelial cells.

The invention further provides cells for use in gene therapy, obtainable by

(a) isolating mononuclear cells from the blood or cell-containing fluids of the body of a mammal;

(b) culturing the cells obtained in step (a) in a cell culture medium comprising one or more of gangliosides, phospholipids, glycolipids and/or growth factors for endothelial cells, including factors influencing differentiation, survival, migration and/or vascularization;

(c) optionally, immortalizing the cells obtained in step (a) or step (b) by transformation with an oncogene, activation of an oncogene, or inactivation of a suppressor gene;

(d) transfecting the cells obtained in step (a) or step (b) or step (c) with a nucleic acid construct for gene therapy, wherein the construct comprises an effector gene which optionally can be activated cell-specifically, cell cycle-specifically, virus-specifically, metabolically, and/or as a result of hypoxia by suitable promoter systems.

As used herein, the phrase “a cell obtainable by” denotes a cell with the same properties as a cell obtained by the listed method, although the cell need not actually have been obtained by the listed method.

Particular embodiments of the invention include a cell as described above, wherein:

the cell is a CD34-, CD14-, CD11-, CD11b-, CD13-, CD64- or CD68-positive cell, or an endothelial cell;

the cell is derived from the blood in veins, capillaries, arteries, umbilical cord or placenta, from the bone marrow, the spleen, the lymph nodes, the peritoneal space, the pleural space, the lymph, the veins, arteries, capillaries and/or the connective tissue fluid;

the growth factor in step (b) is selected from the group comprising ECGF, FGFα, FGFβ, ECAF, IGF-1; IGF-2; Sl-3; EGF; SCF, TGFβ, Tie-2-ligands, stromal derived Factor-1, GM-CSF, G-CSF, M-CSF, Sl-4, Sl-1, CSF-1, Sl-8, PDGF, TFNα, oncostatin M, BG1, platelet derived endothelial cell growth factor, TNFα, angiogenin, pleiotrophin, VEGF and other KDR and Flt ligands, such as VEGF-B, VEGF-C, VEGF-D, and neuropilin, and Flt-3 ligand;

the oncogene in step (c) is mutated such that the oncogene gene product can still completely activate the cell cycle, but this activation of the cell cycle is no longer inhibitable by cellular inhibitors;

the oncogene is selected from the group comprising mutated cdk-4, cdk-6 and cdk-2, and, optionally, the nucleotide sequence for cdk-4 in position 24 is mutated such that the encoded arginine is replaced by a cysteine;

the inactivation of a suppressor gene in step (c) is achieved by transforming the cell with a nucleic acid sequence coding for a protein which inactivates at least one suppressor gene product, and, optionally, the protein inactivating the suppressor gene product is selected from the group comprising the E1A protein of the adenovirus, the E1B protein of the adenovirus, the large T antigen of the SV40 virus, the E6 protein of the papillomavirus, the E7 protein of the papillomavirus, the MDM-2 protein and a protein comprising at least one amino acid sequence LXDXLXXL-II-LXCXEXXXXXSDDE, in which X is a variable amino acid and -II- is any desired amino acid chain of 7-80 amino acids;

the oncogene used for the transformation or the nucleic acid sequence used for the inactivation of the suppressor gene is linked to an endothelium-specific activation sequence which controls the transcription of the oncogene or of the mentioned nucleotide sequence;

the nucleic acid construct in step (d) comprises at least one unrestrictedly activatable, endothelial cell-specific, virus-specific, metabolically activatable and/or cell cycle-specifically activatable activation sequence and at least one effector gene whose expression is controlled by the activation sequence, and, optionally, activation of the activation sequence is self-enhancing and/or pharmacologically controllable;

the expression of the effector gene is controlled by at least two identical or different activation sequences, and, optionally, activation of the activation sequence is self-enhancing and/or pharmacologically controllable;

where a second activator sequence is used, the second activator sequence may be selected from the group comprising promoter sequences of viruses such as HBV, HCV, HSV, HPV, EBV, HTLV, CMV or HIV; promoter or enhancer sequences activated by hypoxia or cell cycle-specific activation sequences of the genes for cdc25C, cdc25B, cyclin A, cdc2, E2F-1, B-myb and DHFR; binding sequences for transcription factors occurring or activated in a cell proliferation-dependent manner, such as monomers or multimers of the Myc E box;

the effector gene codes for an active compound which is selected from the group comprising cytokines, chemokines, growth factors, receptors for cytokines, chemokines or growth factors, proteins having antiproliferative or cytostatic or apoptotic action, antibodies, antibody fragments, angiogenesis inhibitors, peptide hormones, clotting factors, clotting inhibitors, fibrinolytic proteins, peptides or proteins acting on the blood circulation, blood plasma proteins and antigens of infective agents or of cells or of tumors, the selected antigen triggering an immune reaction;

the effector gene codes for an enzyme which cleaves a precursor of a drug (a prodrug) into a drug;

the effector gene codes for a ligand-active compound fusion protein or a ligand-enzyme fusion protein, the ligand being selected from a group comprising cytokines, growth factors, antibodies, antibody fragments, peptide hormones, mediators, cell adhesion proteins and LDL receptor-binding proteins;

the nucleic acid construct introduced into the endothelial cell is DNA; and/or

the nucleic acid construct is inserted in a vector which optionally is a plasmid vector or a viral vector.

In accordance with the present invention, the cells described herein can be administered externally, orally, intravesically, nasally, intrabronchially or into the gastrointestinal tract or injected into an organ, into a body cavity, into the musculature, subcutaneously or into the blood circulation in gene therapy methods for the prophylaxis or therapy of a disorder.

The cells described herein can be used for the production of a therapeutic for the treatment of a disorder selected from the group comprising tumors, leukemias, autoimmune disorders, allergies, arthritides, inflammations, organ rejections, transplants-versus-host reactions, blood clotting disorders, circulation disorders, anemia, infections, hormone disorders and CNS damage.

The invention also comprises a process for the production of the cells described herein, which comprises carrying out the following steps:

(a) isolating cells from the blood or cell-containing fluids of the body;

(b) culturing the cells obtained in step (a) in a cell culture medium comprising gangliosides, phospholipids, glycolipids and/or growth factors;

(c) optionally, immortalizing the cells obtained in step (a) or (b) by transformation with an oncogene, activation of an oncogene or inactivation of a suppressor gene;

(d) transfecting the cells obtained in step (a) and (b) or in step (c) with a nucleic acid construct for gene therapy, comprising an effector gene which can be activated target cell-specifically, cell cycle-specifically, virus-specifically and/or by hypoxia by suitable promoter systems.

The invention also comprises pharmaceutical compositions comprising the cells described above together with a pharmaceutically acceptable vehicle, carrier, or diluent. Suitable vehicles, carriers and diluents are known in the art.

The invention also comprises cells as described herein for the endothelialization of injured vessels.

Further details of the invention, specific embodiments of the invention and corresponding examples are described in what follows.

I. Preparation of Endothelial Cells

1. Isolation and Culturing of Precursor Cells of Endothelial Cells.

The present invention provides a method for isolating precursor cells from endothelial cells. This method is described in detail below.

Cell-containing fluids of the body of a mammal are removed from their respective organs using, for example, invasive procedures known to those skilled in the art. Suitable cell-containing fluids of the body include, for example:

blood obtained from veins, capillaries, arteries or the umbilical cord or placenta; bone marrow cell suspensions; spleen cell suspensions; lymph node cell suspensions; peritoneal cell suspensions; pleural cell suspensions; lymph connective tissue fluid (issuing, for example, from the surface of a superficially, e.g. mechanically, damaged epidermis) or from veins, arteries, or capillaries, or from connective tissue fluid.

Erythrocytes, granulocytes and other cell components are separated from these fluids of the body, for example, by density gradient centrifugation, and platelets are separated, for example, by differential centrifugation, according to methods known to those skilled in the art.

Mononuclear (nucleus-containing) cells, such as cells selected from the group consisting of CD34-, CD14-, CD11-, CD11b-, CD13-, CD64- and CD68-positive cells and endothelial cells, are suspended in serum-containing cell culture medium. In accordance with the present invention, the cell culture medium used contains one or more of gangliosides, phospholipids and growth factors, as discussed in more detail below. Such substances promote the differentiation of mononuclear cells into endothelial-like cells.

In one embodiment of this invention, the isolated, mononuclear (nucleus-containing) cells are cultured in the cell culture medium of the invention and differentiated to give endothelial like cells.

In another embodiment of this invention, the isolated mononuclear (nucleus-containing) cells are incubated with an antibody against a monocyte/macrophage-typical surface marker (for example, CD11, CD11b, CD13, CD14, CD34, CD64, or CD68, which are comercially available, for example, from DAKO, Becton Dickinson, Pharmingen, and Serotec) which is optionally coupled to solid phase particles for separation. For example, the antibodies can be coupled to polysaccharide-coated iron or iron oxide particles, in which case the antibodies/particles are incubated with the cells, the particles are washed, and the cells coated in this way are then recovered with the aid of a magnet. The cells then can be added to the cell culture medium of the present invention, which contains one or more of gangliosides, phospholipids, and growth factors, as discussed below. The cells then can be further proliferated in vitro and differentiated to give endothelial cells. In accordance with this embodiment, the purity and yield of endothelial cells may be increased. After proliferation and/or differentiation, the cells are optionally immortalized and/or transfected or transduced in vitro.

In another embodiment of this invention, the isolated mononuclear (nucleus-containing) cells are preincubated in the cell culture medium of the invention for greater than about 1 hour, for example, for greater than 1 hour, for further differentiation and proliferation. Under these conditions, endothelial precursor cells develop surface markers increasingly typical of monocytes/macrophages (for example, CD11, CD11b, CD13, CD14, CD34, CD64, and CD68). These endothelial precursor cells can be isolated, for example, with the aid of a magnet using an antibody directed against these monocyte markers (e.g., an antibody against CD11 or CD14) and coupled to, for example, dextran-coated iron particles. The cells can be proliferated further in vitro and differentiated to give endothelial cells.

In an alternative embodiment, CD34-positive cells (hematopoietic stem cells) are isolated from nonadherent mononuclear cells such as, for example, described by Asahara et al., Science 275, 964 (1997), and proliferated further in vitro and differentiated to give endothelial cells. As used herein, the phrase “nonadherent mononuclear cells” includes circulating, peripheral cells, such as monocytes.

In another embodiment of the invention, the isolated mononuclear (nucleus-containing) cells are suspended in cell culture medium, for example, in the cell culture medium of the invention, and the remaining phagocytizing cells (e.g. monocytes, macrophages, granulocytes) are removed, for example, by adhering to the surface or by phagocytosis of protein-loaded, dextran-coated iron particles with the aid of a magnet and/or by countercurrent centrifugation according to processes known to those skilled in the art. The remaining mononuclear cells containing CD34-positive cells are cultured in the cell culture medium according to the invention and differentiated to give endothelial cell-like cells. In accordance with this embodiment, the yield of endothelial cells my be increased.

The cell culture medium of the present invention comprise one or more of gangliosides, phospholipids and glycolipids, which may support the differentiation of mononuclear cells into endothelial cells by growth factors. In one particular embodiment of the invention, the cell culture medium comprises one or more growth factors for endothelial cells, such as, for example, growth factors influencing differentiation, survival, migration and vascularization. Examples of suitable growth factors include:

vascular endothelial growth factor (VEGF) and other KDR or Flt ligands, such as VEGF-B, VEGF-C, VEGF-D and neuropilin; fibroblast growth factor (FGFα, FGFβ); epidermal growth factor (EGF); insulin-like growth factor (IGF-1, IGF-2); β-endothelial cell growth factor (ECGF); endothelial cell attachment factor (ECAF); interleukin-3 (IL-3); GM-CSF; G-CSF; M-CSF; interleukin-4 (IL-4); interleukin-1 (IL-1); colony stimulating factor (CSF-1); interleukin-8 (IL-8); platelet derived growth factor (PDGF); interferonγ (IFNγ); oncostatin M; B61; platelet derived endothelial cell growth factor (PDEGF); stem cell factor (SCF); transforming growth factor β (TGF-β); angiogenin; pleiotrophin; Flt-3 ligand (FL); Tie-2-ligands, such as angiopoietin-1; stromal derived factor-1 (SDF-1); TNFα; and midkines.

In one embodiment, the cell culture medium comprises one or more growth factors selected from the group consisting of ECGF, FGFα, FGFβ, VEGF, ECAF, IGF-1, IGF-2, IL-3, EGF, SCF, TGFβ, angiogenin, pleiotrophin and Flt-3 ligand. In another embodiment, the cell culture medium comprises VEGF and bFGF. In another embodiment, the cell culture medium comprises ECGF and VEGF. In another embodiment, the cell culture medium comprises ECGF, VEGF and fetal calf serum.

The cells are grown for a time that can be selected by those skilled in the art, such as, for example, for from between about 6 hours to about 8 weeks, in particular, from 6 hours to 8 weeks. The cells then may be manipulated further according to the invention. For example, the cells can be immortalized or transfected as discussed above, and as explained in more detail below. Alternatively, the endothelial cells can be employed directly, for example, to promote the endothelialization of injured vessels or angiogenesis.

2. Isolation of Endothelial Cells

Endothelial cells suitable for use in accordance with the present invention also can be obtained using methods known to those persons skilled in the art. For example, endothelial cells can be obtained from fatty tissue, by scraping out veins, or by removing umbilical cord endothelium. Endothelial cells obtained by these methods can be cultured as described above in accordance with the invention.

II. Immortalization of Endothelial Cells

In accordance with the present invention, a nucleotide sequence (Component A) for a protein can be inserted into one or more nonadherent mononuclear cells, in particular, endothelial cells according to the invention, which immortalizes the cells. That is, it causes the cells to continuously run through the cell division cycle and thus to become a nonalternating, “permanently” dividing cell line. Such immortalizing nucleotide sequences or genes are known in the art, and include, for example, oncogenes. In accordance with the present invention, the oncogene can be of cellular or viral origin. Examples of cellular oncogenes are comprehensively described by Wynford-Thomas, J. Pathol. 165: 187 (1991); Harrington et al., Curr. Opin. Genet. Developm. 4: 120 (1994); Gonos et al., Anticancer Res. 13: 1117 (1993); and Baserga et al., Cancer Surveys 16: 201 (1993). Oncogenes can be introduced into a cell, for example, an endothelial cell, using methods known to those skilled in the art.

Cells also carry protooncogenes in their genome which, in accordance with the present invention, can be activated in the cell using methods known to those skilled in the art. For example, the protooncogenes can be converted to oncogenes.

In one embodiment of the invention, Component A is a nucleotide sequence which encodes a protein which inactivates the protein of a suppressor gene. Examples of suppressor genes are comprehensively described by Karp and Broder, Nature Med. 4: 309 (1995); Skuse and Ludlow, The Lancet 345: 902 (1995); Duan et al., Science 269: 1402 (1995); Hugh et al., Cancer Res. 55: 2225 (1995); Knudson, P.N.A.S., USA 90: 10914 (1993)).

Examples of genes suitable for use as Component A in accordance with the present invention, i.e., which code for a protein which inactivates the expression product of a suppressor gene include:

| Protein of the | |
|---|---|
| suppressor gene | Gene (Component A) coding for: |
| Retinoblastoma | E1A protein of the adenovirus |
| protein (Rb | (Whyte et al., Nature 334:124 |
| protein) and | (1988)) |
| related proteins, | large T antigen of the SV40 virus |
| such as p107 and | (De Caprio et al., Cell 54 275 |
| p130 | (1988)) |
| | E7 protein of the papilloma virus |
| | (for example, HPV-16, HPV-18) |
| | (Dyson et al., Science 243:934 |
| | (1989)) |
| | a protein comprising the amino |
| | acid sequence LXDXLXXL-II- |
| | LXCXEXXXXXSDDE (SEQ ID NO: 1), in |
| | which X is a variable amino acid |
| | and -II- is any desired amino acid |
| | chain of 7-80 amino acids |
| | (selected from the 20 natural |
| | amino acids occurring in |
| | translation products; Muenger et |
| | al., Cancer Surveys 12:197 (1992)) |
| p53 | E1B protein of the adenovirus |
| | (Sarnow et al., Cell 28:287 |
| | (1982)) |
| | large T antigen of the SV40 virus |
| | (Lane et al., Nature 278:261 |
| | (1979)) |
| | E6 protein of the papilloma virus |
| | (for example, HPV-16, HPV-18) |
| | (Werness et al., Science 248:76 |
| | (1990); Scheffner et al., Cell |
| | 63:1129 (1990)) |
| | MDM-2 protein |
| | (Momand et al., Cell 69:1237 |
| | (1992); Oliner et al., Nature |
| | 362:857 (1993); Kussie et al., |
| | Science 274:948 (1996)) |

In accordance with one embodiment of the present invention, Component A is a mutated nucleotide sequence for a cell cycle regulation protein which is modified by mutation such that it can still fully activate the cell cycle but is no longer subject to inhibition by cellular inhibitors. Examples of such nucleotide sequences include mutated nucleotide sequences coding for cyclin-dependent kinases which retain their kinase activity but have lost the ability to bind to the cellular cdk inhibitors.

Further examples of Component A include:

cdk-4 mutated such that it is no longer inhibited by p16, p15 and/or p21.

cdk-6 mutated such that it is no longer inhibited by p15 and/or p18.

cdk-2 mutated such that it is no longer inhibited by p21, p27 and/or WAF-1.

For example, cdk4 can be mutated by the replacement of an arginine with a cysteine at position 24. Such a mutated cdk4 has kinase activity but is no longer subject to inhibition by pl5 and pl6. (Wölfel et al., Science 269:1281 (1995)).

In another embodiment of the invention, Component A is a transforming gene whose expression is regulated by a self-amplifying promoter element, optionally in combination with a pharmacologically controllable promoter, which is discussed in more detail below.

In another embodiment of this invention, in addition to Component A, a nucleotide sequence which consists of an endothelial cell-specific promoter or enhancer sequence (Component B) is introduced into endothelial cells or, particularly, into endothelial precursor cells or into the cells of a cell mixture containing a proportion of endothelial cells or endothelial precursor cells or a proportion, increased in comparison to the proportion in blood, of CD34-, CD11-, CD11b-, CD14-, CD13-, CD64- and/or CD68-positive cells. In accordance with this embodiment, the transcription of Component A is activated by the binding of the transcription factors of the endothelial cell or of the endothelial precursor cell to Component B.

Thus, in accordance with the present invention, cells can be immortalized by a process selected from the group consisting of transforming the cells with an exogenous oncogene; activating an endogenous oncogene (or protooncogene); and inactivating an endogenous suppressor gene. Where the cell is immortalized using an exogenous oncogene, the exogenous oncogene optionally is linked to an endothelial cell-specific promoter which controls transcription of the oncogene. Where the cell is immortalized by inactivating an endogenous suppressor gene, the nucleotide sequence used to inactivate the suppressor optionally is linked to an endothelial cell-specific promoter which controls transcription of the nucleotide sequence.

In accordance with one embodiment of the invention, a nuclear localization signal (Component C) is used to improve the localization of Component A in the cell nucleus. Component C can be operatively linked or attached to Component A, as shown below:

| Component B | Component A | Component C |
|---|---|---|
| Endothelial cell- | Nucleotide | Nuclear |
| specific promoter | sequence coding | localization |
| or enhancer | for a protein | signal |
| sequence | which leads to | |
| | continual cell | |
| | division | |

By using a nucleic acid construct as shown above, only endothelial cells and endothelial cell precursors contained in a heterogeneous cell mixture in an immortalized stage, i.e., in permanently dividing endothelial and endothelial precursor cells, are transfected (or transduced), so that after a period of time, such as, for example, a few days, the transfected endothelial or endothelial precursor cells proportionately dominate the cell culture, and after a time which is dependent on the culture conditions, but which will be readily apparent to those skilled in the art, the transfected endothelial or endothelial precursor cells will be present exclusively in the cell culture.

It is therefore possible, using these nucleic acid constructs and the processes of the present invention, to prepare large amounts of homogeneous endothelial cells in a relatively short time with relatively low cost and little starting materials even if only a few endothelial or endothelial precursor cells are available as starting material, and even if the cells are present in a heterogeneous cell mixture. The endothelial and endothelial precursor cells are useful for gene therapy methods of prophylaxis or treatment of disorders when further transduced with an effector gene, as described below. Such cells also are useful in in vitro pharmacological studies.

III. Preparation of Genetically Modified Endothelial Cells for Prophylaxis and/or Therapy

The present invention provides a nucleic acid construct for transfecting endothelial cells or endothelial precursor cells to make cells capable of expressing biologically active proteins, where the biologically active protein is useful for the prophylaxis or therapy of a disorder. This construct comprises a gene for the biologically active protein (an effector gene) (Component E) and can be introduced into endothelial cells such as endothelial cells obtained by the methods of the present invention described above. The nucleic acid construct may further comprise a promoter (Component D) operably linked to the gene for the biologically active protein. In one embodiment, the nucleic acid construct for transfecting cells contains at least Component D and Component E. As discussed in more detail below, the promoter can operate cell-specifically, cell cycle specifically, virus specifically, metabolically or by hypoxia. Additionally, the promoter may be inducible. In accordance with one embodiment, the nucleic acid construct comprises at least two promoters operably linked to the gene for the biologically active protein, which promoters may be the same or different.

1. Selection of Promoter Sequences

In accordance with the present invention, nucleotide sequences suitable for use as promoter sequences are those which, after binding transcription factors, activate the transcription of a transgene adjacently placed at the 3′ end, such as, for example, a structural gene, in particular, an effector gene (Component E). In accordance with the present invention, at least one of Component B and Component D may comprise an endothelial cell-specific promoter sequence. As described above, these promoter sequences can be inserted into the endothelial cell or endothelial precursor cell. The endothelial cell-specific promoter sequence can be combined with one or more additional promoter sequences. The choice of the promoter sequence(s) to be combined with the endothelial cell-specific promoter depends on the disorder to be treated, and the selection of suitable promoters is well-within the capabilities of the skilled artisan.

In accordance with one embodiment of the invention, the additional promoter sequence is induced unrestrictedly, cell-specifically, in particular, endothelial cell-specifically, under certain metabolic conditions, such as, for example, by hypoxia, or is induced or switched off by a drug. Alternatively or additionally, the promoter may be activated virus-specifically and/or cell cycle-specifically. Promoters of this type are described in the following patent applications: EP95931204.2; EP95930524.4; EP95931205.9; EP95931933.6; EP96110962.2; DE19704301.1, EP97101507.8; EP97102547.3; DE19710643.9 and EP97110995.8, which are incorporated herein by reference in their entirety. Suitable promoter sequences include, for example:

unrestrictedly activatable promoters and activator sequences, such as, for example, the promoter of RNA polymerase III, the promoter of RNA polymerase II, the CMV promoter and enhancer, and the SV40 promoter.

metabolically activatable promoter and enhancer sequences, such as, for example, an enhancer inducible by hypoxia (Semenza et al., P.N.A.S. 88: 5680 (1991); McBurney et al., Nucl. Acids Res. 19: 5755 (1991)).

cell cycle-specifically activatable promoters, such as, for example, the promoter of the cdc25B gene, the cdc25C gene, the cyclin A gene, the cdc2 gene, the B-myb gene, the DHFR gene, or the E2F-1 gene; binding sequences for transcription factors occurring or activated during cell proliferation, including, for example, binding sequences for c-myc proteins. Further examples of binding sequences include monomers or multimers of the nucleotide sequence designated as Myc E box (5′-GGAAGCAGACCACGTGGTCTGCTTCC-3′ (SEQ ID NO:2), Blackwood and Eisenmann, Science 251: 1211 (1991)).

self-enhancing and/or pharmacologically controllable promoters. In the simplest case, where a combination of identical or different promoters is used, one promoter is inducible, for example, it is a promoter which can be activated or switched off by tetracycline, such as the tetracycline operator in combination with an appropriate repressor. In an alternative embodiment, the promoter, is self-enhancing with or alternatively without a pharmacologically controllable promoter unit. Suitable self-enhancing and/or pharmacologically controllable promoters are described in Patent Application DE19651443.6, which is incorporated herein by reference in its entirety.

endothelial cell-specifically activatable promoters, including promoters or activator sequences of promoters or enhancers of those genes which code for proteins preferably formed in endothelial cells. Promoters of the genes for the following proteins are suitable for use in accordance with the present invention:

brain-specific, endothelial glucose-1-transporter; endoglin; VEGF receptor 1 (flt-1); VEGF receptor 2 (flk-1, KDR); tie-1 or tie-2; B61 receptor (Eck receptor); B61; endothelin, in particular, endothelin B or endothelin-1; endothelin receptors, in particular, the endothelin B receptor; mannose-6-phosphate receptors; von Willebrand factor; IL-1α; IL-1β; IL-1 receptor; vascular cell adhesion molecule (VCAM-1); interstitial cell adhesion molecule (ICAM-3); synthetic activator sequences;

Alternatives to natural endothelial cell-specific promoters also can be used in accordance with the invention, such as synthetic activator sequences which consist of oligomerized binding sites for transcription factors which are preferentially or selectively active in endothelial cells. One example of these is the transcription factor GATA-2, whose binding site in the endothelin-1 gene is 5′-TTATCT-3′ (Lee et al., Biol. Chem. 16188 (1991); Dormann et al., J. Biol. Chem. 1279 (1992); Wilson et al., Mol. Cell Biol. 4854 (1990)).

The identical or different promoters can be combined, for example, by successive linkage of the promoters in the reading direction from 5′ to 3′ of the nucleotide sequence. Patent Applications GB9417366.3; EP97101507.8; EP97102547.3; DE19710643.9; DE19617851.7; DE19639103.2 and DE19651443.6, which are incorporated herein by reference in their entirety, describe technologies which are preferably employed to combine promoters. Examples of technologies of this type are set forth below.

Chimeric Promoters

A chimeric promoter is a combination of a cell-specifically, metabolically or virus-specifically activatable activator sequence located upstream of a promoter module. An example is the chimeric promoter containing the nucleotide sequence CDE-CHR or E2FBS-CHR₁ to which suppressive proteins bind, thereby inhibiting the activation in the G₀ and G₁ phase of the cell cycle of the activator sequence located upstream. GB9417366.3; Lucibello et al., EMBO J., 12 (1994).

Continuing investigations on the manner of functioning of the promoter element CDE-CHR have shown that cell cycle-dependent regulation by the CDE-CHR element of an activator sequence located upstream is largely dependent on the activation sequence of transcription factors being activated by glutamine-rich activation domains. Zwicker et al., Nucl. Acids Res., 3822 (1995). Transcription factors of this type include, for example, Spl and NF-YA. This consequently restricts the use of the promoter element CDE-CHR for chimeric promoters. The same is to be assumed for the promoter element E2F-BS-CHB of the B-myb gene. Zwicker et al., Nucl. Acids Res. 23, 3822 (1995).

Hybrid Promoters

Suitable hybrid promoters are described in Patent Application DE19639103.2. In accordance with the embodiment of the present invention where an endothelial cell-specific promoter is combined with at least one additional promoter, a gene construct containing the following components may be selected:

The nucleotide sequence of the endothelial cell-specific promoter in a form in which at least one binding site for a transcription factor is mutated to block initiation of the transcription of the effector gene.

A transgene, in particular a structural gene (referred to herein as an effector gene), which codes for a biologically active protein for the prophylaxis or therapy of a disorder, as mentioned above and discussed in more detail below.

At least one additional promoter or enhancer sequence which is activatable unspecifically, cell-specifically, virus-specifically, by tetracycline and/or cell cycle-specifically, which activates the transcription of at least one gene for at least one transcription factor, which is mutated such that it can bind to the mutated binding site(s) in the endothelial cell-specific promoter and can activatethe endothelial cell-specific promoter.

In an exemplary embodiment of this invention, it is possible to show the mutation in the promoter sequence, for example a mutation of the TATA box of the cdc25B promoter. The mutation of the TATA can, for example, be TGTATAA. By means of this mutation, the DNA-binding site of the normal TATA box-binding protein (TBP) is no longer recognized and the effector gene can no longer be efficiently transcribed. Accordingly, the nucleic acid sequence which codes for the TBP must have a co-mutation. By means of this co-mutation, the TBP binds to the mutated TATA box (e.g., to TGTATAA) and thus leads to the efficient transcription of the effector gene. Co-mutations of the TBP gene of this type are described, for example, by Strubin and Struhl, Cell, 721 (1992); Heard et al. EMBO J., 3519 (1993).

Multiple Promoters in Combination with a Nuclear Retention Signal and a Nuclear Export Factor

This technology is described in detail in Patent Application DE19617851.7, which is incorporated herein by reference in its entirety. In accordance with the invention, a promoter of this type may contain, for example, the following components:

a first endothelial cell-specific, activatable promoter or enhancer sequence, which activates the basal transcription of the transgene described below;

a transgene, in particular a structural gene (an effector gene) coding for an active compound useful for the prophylaxis or therapy of a disorder, as mentioned above and discussed in more detail below.

a nuclear retention signal (NRS), whose cDNA is linked indirectly or directly at the 5′ end to the 3′ end of the structural gene (b). In accordance with one embodiment, the transcription product of the nuclear retention signal has a binding structure for a nuclear export factor.

a non-specifically, cell-specifically, virus-specifically, metabolically and/or cell cycle-specifically activatable promoter or enhancer sequence which activates the basal transcription of a nuclear export factor.

a nucleic acid coding for a nuclear export factor (NEF) which binds to the transcription product of the nuclear retention signal thereby mediating the transport of the transcription product of the transgene from the cell nucleus.

In accordance with one embodiment of the invention, the gene coding for the nuclear retention signal is selected from the group consisting of the Rev-responsive element (RRE) of HIV-1 or HIV-2, the RRE-equivalent retention signal of retroviruses or the RRE-equivalent retention signal of HBV.

In accordance with one embodiment of the invention, the nuclear export factor is a gene selected from the group comprising the Rev gene of the viruses HIV-1, HIV-2, maedi-visna virus, caprine arthritis encephalitis virus, equine infectious anemia virus, feline immunodeficiency virus, the Rev gene of retroviruses, of HTLV or the gene of the hnRNP-A1 protein or the gene of the transcription factor TFIII-A.

Activator-responsive Promoter Units

Activator-responsive promoter units are described in detail in Patent Application DE19617851.7, which is incorporated herein by reference in its entirety. In accordance with the present invention, an activator-responsive promoter unit may comprise the following components:

one or more identical or different promoter or enhancer sequences, which may be activatable cell cycle-specifically, cell proliferation-dependently, metabolically, endothelial cell-specifically or virus-specifically or both cell cycle-specifically and metabolically, endothelial cell-specifically or virus-specifically (i.e., so-called chimeric promoters);

one or more identical or different activator subunits which are located downstream of the promoter or enhancer sequences and which are activated by the promoter or enhancer sequences in their basal transcription;

an activator-responsive promoter which is activated by the expression products of one or more activator subunits.

In accordance with one embodiment of the invention, the activator-responsive promoter unit consists of the promoter or enhancer sequences, the activator subunits and the activator-responsive promoter described above.

In accordance with one embodiment of the invention, activator-responsive promoter units are binding sequences for chimeric transcription factors from DNA-binding domains, protein-protein interaction domains or transactivation domains.

The transcription factor binding sites mentioned herein can be present singly (monomers) or in a multiple copies (multimers), for example, of up to 10 copies).

An example of an activator-responsive promoter activated by two activator subunits is the LexA operator in combination with the SV40 promoter. The first activator subunit of this promoter comprises the cDNA for the LexA-DNA binding protein coding for amino acids 1-81 or 1-202, whose 3′ end is linked to the 5′ end of the cDNA for the Gal80 protein (amino acids 1-435). The second activator subunit comprises the cDNA of the Gal80 binding domain of the Gal4 protein coding for amino acids 851-881, whose 3′ end is linked to the 5′ end of the cDNA of the SV40 large T antigen coding for amino acids 126-132, whose 3′ end is linked to the 5′ end of the cDNA for the transactivation domain of the VP16 of HSV-1 coding for amino acids 406-488. This promoter is suitable for use in accordance with the present invention.

A further example of an activator-responsive promoter activated by two activator subunits is the binding sequence of the Gal4 protein in combination with the SV40 promoter. The first activation unit of this promoter comprises the cDNA for the DNA binding domain of the Gal4 protein (amino acids 1-147) whose 3′ end is linked to the 5′ end of the cDNA for the Gal80 protein (amino acids 1-435). The second activation subunit of this promoter comprises the cDNA for the Gal80 binding domain of Gal4 (amino acids 851-881) whose 3′ end is linked to the 5′ end of the cDNA of the nuclear localization signal of SV40 (SV40 large T; amino acids 126-132), whose 3′ end is linked to the 5′ end of the cDNA for the transactivation domain of the VP16 of HSV-1 coding for the amino acids 406-488. This promoter is suitable for use in accordance with the present invention.

A further example of two-activator subunits which activate the activator-responsive promoter consisting of the binding sequence for the Gal4 protein and the SV40 promoter comprises a first activation unit which comprises the cDNA for the cytoplasmic domain of the CD4 T-cell antigen (amino acids 397-435) whose 5′ end is linked to the 3′ end of the cDNA for the transactivation domain of the VP16 of HSV-1 (amino acids 406-488), whose 5′ end is in turn linked to the 3′ end of the cDNA of the nuclear localization signal of SV40 (SV40 large T; amino acids 16-132) and the second activation unit comprises the cDNA of the nuclear localization signal of SV40 (SV40 large T; amino acids 126-132), the cDNA for the DNA binding domain of the Gal4 protein (amino acids 1-147) whose 3′ end is linked to the 5′ end of the cDNA for the CD4 binding sequence of the p56 lck protein (amino acids 1-71).

IV. Selection of the Effector Gene

As described above, the present invention provides a nucleic acid construct comprising an effector gene (a transgene) (Component E), which is a gene for a biologically active protein, for transfecting endothelial cells or endothelial precursor cells to make cells capable of expressing a biologically active protein, useful, for example in gene therapy methods for the prophylaxis or therapy of disorders.

The transfected cells may be administered externally, orally, intravesically, nasally, intrabronchially, subcutaneously, into the gastrointestinal tract, or are injected into an organ, into a body cavity, into musculature, or into blood circulation.

Examples of disorders that can be treated with the cells include leukemias, autoimmune disorders, allergies, arthritides, inflammations, organ rejections, transplant-host reactions, blood clotting disorders, circulation disorders, anemia, infections, hormone disorders and central nervous system (CNS) damage.

The effector gene (Component E) codes for a biologically active protein that is useful in the prophylaxis and/or therapy of a disorder. For example, the active compound may be selected from the group consisting of enzymes, cytokines, growth factors, antibodies or antibody fragments, receptors for cytokines or growth factors, proteins having antiproliferative, apoptotic or cytostatic action, angiogenesis inhibitors, clotting inhibitors, substances having fibrinolytic activity, plasma proteins, complement-activating proteins, peptide hormones, virus coat proteins, bacterial antigens, parasitic antigens, proteins acting on the blood circulation and ribozymes.

In accordance with one embodiment of the invention, the effector gene is a structural gene which codes for a ribozyme which inactivates the mRNA which codes for a protein selected from the group consisting of cell cycle control proteins, such as, for example, cyclin A, cyclin B, cyclin D1, cyclin E, E2F1-5, cdc2, cdc25C and DP1, virus proteins, cytokines, growth factors, and their receptors. In a further embodiment, the effector gene codes for an enzyme which cleaves a precursor of a drug (a prodrug) into a drug.

In accordance with another embodiment, the effector gene codes for a ligand-effector fusion protein, where the ligand is, for example, an antibody, an antibody fragment, a cytokine, a growth factor, an adhesion molecule or a peptide hormone, and the effector is, for example, a pharmacologically active compound such as those described above, or an enzyme. For example, the structural gene can code for a ligand-enzyme fusion protein, where the enzyme cleaves a precursor of a drug into a drug and the ligand binds to a cell surface, such as to endothelial cells or tumor cells.

The choice of the effector gene and of the further promoter element optionally to be combined with the endothelial cell-specific promoter depends on the prophylaxis and/or therapy of the particular disorder, and is well-within the capabilities of the skilled artisan. Examples of suitable combinations of promoter sequences and effector genes are set forth below. See also the Patent Applications EP97101507.8; EP97102547.3; DE19710643.9; DE197704301.1; DE19617851.7; DE19639103.2; DE19651443.6; EP95931204.2; EP95930524.4; EP95931205.9; EP95931933.6 and DE19701141.1, which are incorporated herein by reference in their entirety.

Therapy of Tumors

Promoters: unspecifically, cell cycle-specifically and metabolically activatable promoters are suitable.

Effector Genes:

(1) genes for inhibitors of cell proliferation, for example, inhibitors of:

the retinoblastoma protein (pRb=p110) or the related p107 and p130 proteins. The retinoblastoma protein (pRb/p110) and the related p107 and p130 proteins can be inactivated, for example, by phosphorylation. In accordance with one embodiment of the invention, the cell cycle inhibitors have mutations for the inactivation sites of the expressed proteins, without these thereby being impaired in their function. Examples of these mutations are described for p10. The DNA sequence for the p107 protein or the p130 protein is mutated analogously.

The p53 protein. The p53 protein is inactivated in the cell either by binding to specific proteins, such as, for example, MDM2, or by oligomerization of p53 via the dephosphorylated C-terminal serine. In accordance with one embodiment, a DNA sequence for a p53 protein is used which is truncated at the C-terminal by serine 392.

p21 (WAF-1); p16 protein; other cdk inhibitors; GADD45 protein; bak protein.

(2) genes for coagulation-inducing factors and angiogenesis inhibitors, for example:

plasminogen activator inhibitor-1 (PAI-1); PAI-2; PAI-3; angiostatin; interferons (IFNα, IFNβ or IFNγ); platelet factor 4; TIMP-1; TIMP-2; TIMP-3; leukemia inhibitory factor (LIF); and tissue factor (TF) and its fragments having clotting activity; factor X or mutations of factor X (see, for example, Patent Application D19701141.1, which is incorporated herein by reference in its entirety).

(3) genes for cytostatic and cytotoxic proteins, for example, genes for:

perforin; granzyme; IL-2; IL-4; IL-12; interferons, such as, for example, IFN-α, IFNβ or IFNγ; TNF, such as TNFα or TNFβ; oncostatin M; sphingomyelinase; and magainin and magainin derivatives. (4) genes for cytostatic or cytotoxic antibodies and for fusion proteins between antigen-binding antibody fragments with cytostatic, cytotoxic or inflammatory proteins or enzymes.

Exemplary cytostatic or cytotoxic antibodies include those directed against membrane structures of endothelial cells such as described, for example, in Burrows et al., Pharmac. Ther. 64: 155 (1994); Hughes et al., Cancer Res. 49: 6214 (1989); Maruyama et al., P.N.A.S., USA 87: 5744 (1990). In particular, these include antibodies against the VEGF receptors. Also suitable are cytostatic or cytotoxic antibodies directed against membrane structures on tumor cells. Antibodies of this type are comprehensively described, for example, in Sedlacek et al., Contrib. to Oncol. 32, Karger Verlag, Munich (1988), and Contrib. to Oncol. 43, Karger Verlag, Munich (1992). Further examples are antibodies against sialyl Lewis; against peptides on tumors, which are recognized by T cells; against proteins expressed by oncogenes; against gangliosides such as GD3, GD2, GM2, 9-O-acetyl GD3, fucosyl GM1; against blood group antigens and their precursors; against antigens on the polymorphic epithelial mucin; and against antigens on heat shock proteins. Antibodies directed against membrane structures of leukemia cells also are suitable. A large number of monoclonal antibodies of this type have been described for diagnostic and therapeutic procedures. Kristensen, Danish Medical Bulletin 41: 52 (1994); Schranz, Therapia Hungarica 38: 3 (1990); Drexler et al., Leuk. Res. 10: 279 (1986); Naeim, Dis. Markers 7: 1 (1989); Stickney et al., Curr. Opin. Oncol. 4: 847 (1992); Drexler et al., Blut 57: 327 (1988); Freedman et al., Cancer Invest. 9: 69 (1991)). Suitable ligands depend on the type of leukemia, and include, for example, monoclonal antibodies or antigen-binding antibody fragments directed against the following membrane antigens:

| Cells | Membrane antigen |
|---|---|
| AML | CD13 |
| | CD15 |
| | CD33 |
| | CAMAL |
| | Sialosyl-Le |
| B-CLL | CDS |
| | CD1c |
| | CD23 |
| | Idiotypes and isotypes of the |
| | membrane immunoglobulins |
| T-CLL | CD33 |
| | M38 |
| | IL-2 receptors |
| | T-cell receptors |
| ALL | CALLA |
| | CD19 |
| | Non-Hodgkin lymphoma |

The humanization of murine antibodies and the preparation and optimization of the genes for Fab and recombinant Fv fragments can be carried out according to techniques known to those skilled in the art. See, e.g., Winter et al., Nature 349: 293 (1991); Hoogenbooms et al., Rev. Tr. Transfus. Hemobiol. 36: 19 (1993); Girol. Mol. Immunol. 28: 1379 (1991); Huston et al., Intern. Rev. Immunol. 10: 195 (1993). The fusion of recombinant Fv fragments with genes for cytostatic, cytotoxic or inflammatory proteins or enzymes likewise can be carried out according to methods known in the art.

(5) genes for fusion proteins of endothelial cell- or tumor cell-binding ligands with cytostatic and cytotoxic proteins or enzymes.

These ligands include, for example, all substances which bind to membrane structures or membrane receptors on endothelial cells. For example, antibodies or antibody fragments; cytokines such as, for example, IL-1 or growth factors or their fragments or partial sequences of them which bind to receptors expressed by endothelial cells, such as, for example, PDGF, bFGF, VEGF, TGF; adhesion molecules which bind to activated and/or proliferating endothelial cells, such as SLex, LFA-1, MAC-1, LECAM-1, VLA-4 or vitronectin; substances which bind to membrane structures or membrane receptors of tumor or leukemia cells, such as growth factors or fragments thereof or partial sequences of them which bind to receptors expressed by leukemia cells or tumor cells. Growth factors of this type are described in Cross et al., Cell 64: 271 (1991); Aulitzky et al., Drugs 48: 667 (1994); Moore, Clin. Cancer Res. 1: 3 (1995); Van Kooten et al., Leuk. Lymph. 12: 27 (1993). The fusion of the genes of these ligands binding to the target cell with cytostatic, cytotoxic or inflammatory proteins or enzymes can be carried out according to methods known in the prior art.

(6) genes for inflammation inducers, for example for:

IL-1; IL-2; RANTES (MCP-2); monocyte chemotactic and activating factor (MCAF); IL-8; macrophage inflammatory protein-1 (MIP-1α, -β); neutrophil activating protein-2 (NAP-2); IL-3; IL-5; human leukemia inhibitory factor (LIF); IL-7; IL-5; eotaxin; IL-13; GM-CSF; G-CSF; M-CSF; cobra venom factor (CVF) or partial sequences of CVF which correspond functionally to the human complement factor C3b, i.e. which, when combined to the complement factor B and, after cleavage by factor D, produce a C3 convertase; the human complement factor C3 or its subsequence C3b; cleavage products of the human complement factor C3, which are functionally and structurally similar to CVF; bacterial proteins which activate complement or cause inflammations, such as, for example, porines of Salmonella typhimurium; “clumping” factors of Staphylococcus aureus; modulins, particularly of gram-negativen bacteria; “Major outer membrane protein” of Legionella or of Haemophilus influenza type B or of Klebsiella, and M molecules of streptococci group G.

(7) genes for enzymes for the activation of precursors of cytostatics, for example for:

enzymes which cleave inactive preliminary substances (prodrugs) into active cytostatics (drugs). Substances of this type and the associated prodrugs and drugs in each case are comprehensively described by Deonarain et al., Br. J. Cancer, 70: 786 (1994); Mullen, Pharmac. Ther. 63: 199 (1994); Harris et al., Gene Ther. 1: 170 (1994). For example, the DNA sequence of one of the following enzymes may be used: herpes simplex virus thymidine kinase; varicella zoster virus thymidine kinase; bacterial nitroreductase; bacterial β-glucuronidase; vegetable β-glucuronidase from Secale cereale; human β-glucuronidase; human carboxypeptidase (CB) for example CB-A of the mast cell, CB-B of the pancreas or bacterial carboxypeptidase; bacterial β-lactamase; bacterial cytosine deaminase; human catalase or peroxidase; phosphatase, in particular human alkaline phosphatase, human acidic prostate phosphatase or type 5 acidic phosphatase; oxidase, in particular human lysyl oxidase or human acidic D-amino-oxidase; peroxidase, in particular human glutathione peroxidase, human eosinophil peroxidase or human thyroid gland peroxidase; galactosidase.

Therapy of Autoimmune Disorders and Inflammations

Promoters: unspecifically-, cell cycle-specifically or metabolically activatable promoters are suitable.

Effector Genes:

(1) genes for the therapy of allergies, for example, genes for IFNβ; IFNγ; IL-10; antibodies or antibody fragments against IL-4; soluble IL-4 receptors; IL-12; TGFβ.

(2) genes for preventing the rejection of transplanted organs, for example, genes for IL-10; TGFJ3; soluble IL-1 receptors; soluble IL-2 receptors; IL-1 receptor antagonists; soluble IL-6 receptors; immunosuppressant antibodies or their V_{H} and V_{L} containing fragments or their V_{H} and V_{L} fragments bonded via a linker. Immunosuppressant antibodies include, for example, antibodies specific for the T-cell receptor or its CD3 complex, against CD4 or CD8, additionally against the IL-2 receptor, IL-1 receptor or IL-4 receptor or against the adhesion molecules CD2, LFA-1, CD28 or CD40.

(3) genes for the therapy of antibody-mediated autoimmune disorders, for example, genes for TGFβ; IFNα; IFNβ; IFNγ; IL-12; soluble IL-4 receptors; soluble IL-6 receptors; immunosuppressant antibodies or their V_{H}and V_{L} containing fragments.

(4) genes for the therapy of cell-mediated autoimmune disorders, for example, genes for IL-6; IL-9; IL-10; IL-13; TNFα or TNFβ; an immunosuppressant antibody or its V_{H} and V_{L} containing fragments.

(5) genes for inhibitors of cell proliferation, cytostatic or cytotoxic proteins, inflammation inducers and enzymes for the activation of precursors of cytostatics. Examples of genes coding for proteins of this type are set forth above.

As described above, effector genes can be used which code for fusion proteins of antibodies or Fab or recombinant Fv fragments of these antibodies or other ligands specific for the target cell and the above-mentioned cytokines, growth factors, receptors, cytostatic or cytotoxic proteins and enzymes.

(6) genes for the therapy of arthritis, such as genes whose expressed protein directly or indirectly inhibits inflammation, for example, inflammation in joints, and/or promotes reconstitution of the extracellular matrix (including cartilage and connective tissue) in joints. Suitable genes include genes for the following: IL-1 receptor antagonist (IL-1-RA), which inhibits the binding of IL-1α, β; soluble IL-1 receptor, which binds and inactivates IL-1; IL-6, which increases the secretion of TIMP and superoxides and reduces the secretion of IL-1 and TNFα by synovial cells and chondrocytes; soluble TNF receptor, which binds and inactivates TNF; IL-4, which inhibits the formation and secretion of IL-1, TNFα and MMP; IL-10, which inhibits the formation and secretion of IL-1, TNFα and MMP and increases the secretion of TIMP; insulin-like growth factor (IGF-1), which stimulates the synthesis of extracellular matrix; TGFβ, especially TGFβ1 and TGFβ2, which stimulates the synthesis of the extracellular matrix; superoxide dismutase; and TIMP, especially TIMP-1, TIMP-2 or TIMP-3.

Therapy of Defective Formation of Blood Cells

Promoters: unspecifically, cell cycle-specifically, and metabolically activatable promoters are suitable.

Effector Genes:

(1) genes for the therapy of anemia, for example, genes for erythropoietin.

(2) genes for the therapy of leukopenia, for example, genes for G-CSF; GM-CSF; M-CSF.

(3) genes for the therapy of thrombocytopenia, for example, genes for IL-3; leukemia inhibitory factor (LIF); IL-11; thrombopoietin.

Therapy of Damage to the Nervous System

Promoters: unspecifically, cell cycle-specifically, and metabolically activatable promoters are suitable.

Effector Genes:

(1) genes for neuronal growth factors, for example, genes for FGF; nerve growth factor (NGF); brain-derived neurotrophic factor (BDNF); neurotrophin-3 (NT-3); neurotrophin-4 (NT-4); ciliary neurotrophic factor (CNTF).

(2) genes for enzymes, for example, genes for tyrosine hydroxylase; and dopa decarboxylase.

(3) genes for cytokines and their inhibitors, which inhibit or neutralize the neurotoxic action of TNFα, for example, genes for TGFβ; soluble TNF receptors, which neutralize TNFα; IL-10, which inhibits the formation of IFNγ, TNFα, IL-2 and IL-4; soluble IL-1 receptors, such as IL-1 receptor I and IL-1 receptor II, which neutralize the activity of IL-1; IL-1 receptor antagonist; and soluble IL-6 receptors.

Therapy of Disorders of the Blood Clotting and Blood Circulation System

Promoters: cell cycle-specifically, cell-unspecifically, and metabolically activatable promoters are suitable.

Effector genes:

(1) genes for the inhibition of clotting or for the promotion of fibrinolysis, for example, genes for tissue plasminogen activator (tPA); urokinase-type plasminogen activator (uPA); hybrids of tPA and uPA; protein C; hirudin; serine proteinase inhibitors (serpines), such as, for example, C-1S inhibitor, α1-antitrypsin or antithrombin III; and tissue factor pathway inhibitor (TFPI).

(2) genes for the promotion of clotting, for example, genes for F VIII; F IX; von Willebrand factor; F XIII; PAI-1; PAI-2; tissue factor and fragments thereof.

(3) genes for angiogenesis factors, for example, genes for VEGF; FGF;

(4) genes for lowering blood pressure, for example, genes for kallikrein and endothelial cell “nitric oxide synthase.”

(5) genes for the inhibition of the proliferation of smooth muscle cells after injuries to the endothelial layer, for example, genes for an antiproliferative, cytostatic or cytotoxic protein or an enzyme for the cleavage of precursors of cytostatics (prodrugs) into cytostatics (drugs) as already mentioned above (under tumor), or a fusion protein of one of these active proteins with a ligand, for example an antibody or antibody fragment specific for muscle cells.

(6) genes for blood plasma proteins, for example, genes for albumin; C1 inactivator; serum cholinesterase; transferrin; and 1-antritrypsin.

Vaccinations

Promoters: unspecific and cell cycle-specific promoters are suitable.

Effector Genes:

(1) genes for the prophylaxis of infectious diseases. Technology for DNA vaccines has been developed. Some DNA vaccines known in the art are not as effective as desired. Fynan et al., Int. J. Immunopharm. 17: 79 (1995); Donnelly et al., Immunol. 2: 20 (1994). In accordance with the present invention, a greater efficacy of the DNA vaccines is to be expected because the vaccine antigen is expressed by endothelial cells. The gene used in accordance with this embodiment is the DNA of a protein formed by the infectious agent. When expressed, the protein triggers an immune reaction, i.e. by antibody binding and/or by cytotoxic T lymphocytes, which leads to the neutralization and/or to destruction of the causative agent. Neutralization antigens of this type have been used as vaccine antigens. See Ellis, Adv. Exp. Med. Biol. 327: 263 (1992).

DNA coding for neutralization antigens of the following causative agents is suitable for use in accordance with the invention: influenza A virus; HIV; rabies virus; HSV (herpes simplex virus); RSV (respiratory syncytial virus); parainfluenza virus; rotavirus; VzV (varicella zoster virus); CMV (cytomegalovirus); measles virus; HPV (human papillomavirus); HBV (hepatitis B virus); HCV (hepatitis C virus); HDV (hepatitis D virus); HEV (hepatitis E virus); HAV (hepatitis A virus); vibrio cholera antigen; Borrelia burgdorf eri; Helicobacter pylori; and malaria antigen. Also suitable are the DNA for an antiidiotype antibody or its antigen-binding fragments, whose antigen binding structures (the “complementary determining regions”) produce copies of the protein or carbohydrate structure of the neutralization antigen of the infective agent. Antiidiotype antibodies of this type can replace carbohydrate antigens in bacterial infective agents. Antiidiotypic antibodies of this type and their cleavage products are comprehensively described in Hawkins et al., J. Immunother. 14: 273 (1993); Westerink and Apicella, Springer Seminars in Immunopathol., 15: 227 (1993).

(2) effector genes for tumor vaccines, including antigens on tumor cells. Antigens of this type are described comprehensively, for example, in Sedlacek et al., Contrib. to Oncol. 32, Karger Verlag, Munich (1988) and Contrib. to Oncol 43, Karger Verlag, Munich (1992). Further examples are the genes for the following antigens and antiidiotype antibodies: sialyl Lewis peptides on tumors, which are recognized by T cells; proteins expressed by oncogenes; blood group antigens and their precursors; antigens on the polymorphic epithelial mucin; and antigens on heat shock proteins.

Therapy of Chronic Infectious Diseases

Promoters: virus-specific, cell cycle-specific, and unspecific promoters are suitable.

Effector Genes:

(1) genes for a protein which has cytostatic, apoptotic or cytotoxic effects.

(2) genes for an enzyme which cleaves a precursor of an antiviral or cytotoxic substance into the active substance.

(3) genes for antiviral proteins, including cytokines and growth factors having antiviral activity, such as, for example, IFNα, IFNβ, IFN-γ, TNFβ, TNFα, IL-1 or TGFβ; antibodies of a specificity which inactivates the respective virus or its V_{H} and V_{L}-containing fragments or produces its V_{H} and V_{L} fragments bonded via a linker as described above. Antibodies against virus antigen include, for example, anti-HBV; anti-HCV; anti-HSV; anti-HPV; anti-HIV; anti-EBV; anti-HTLV; anti-Coxsackie virus; and anti-Hantaan virus. An anti-HIV Rev-binding protein also is suitable. These proteins bind to the Rev RNA and inhibits Rev-dependent posttranscriptional stages of retroviral gene expression. Examples of Rev-binding proteins include RBP9-27; RBP1-8U; RBP1-8D; pseudogenes of RBP1-8. Genes for ribozymes which digest the mRNA of genes for cell cycle control proteins or the mRNA of viruses also are suitable. Ribozymes catalytic for HIV are described comprehensively, for example, in Christoffersen et al., J. Med. Chem. 38: 2033 (1995).

(4) genes for antibacterial proteins, including, for example, antibodies which neutralize bacterial toxins or opsonize bacteria, for example, antibodies against meningococci C or B; coli; Borrelia; Pseudomonas; Helicobacter pylori; and Staphylococcus aureus. The invention also encompasses a nucleic acid construct comprising a combination of two or more effector genes described above. The effector genes may be the same or different and may code for the same or different proteins. The construct may further comprise a promoter or, in accordance with one embodiment, the cDNA of an “internal ribosome entry site” (IRES), connected as a regulator element between the two structural genes. The promoter or IRES facilitates expression of both effector genes.

An IRES makes possible the expression of two DNA sequences connected to one another via an IRES. IRESs of this type have been described, for example, by Montford and Smith, TIG 11: 179 (1995); Kaufman et al., Nucl. Acids Res. 19: 4485 (1991); Morgan et al., Nucl. Acids Res. 20: 1293 (1992); Dirks et al., Gene 128: 247 (1993); Pelletier and Sonenberg, Nature 334: 320 (1988) and Sugitomo et al., BioTechn. 12: 694 (1994). For example, the cDNA of the IRES sequence of the poliovirus (position≦140 to ≧630 of the 5′ UTR) can be used.

In accordance with one embodiment of the invention, structural genes (i.e., effector genes) which have an additive action are linked via a promoter or an IRES sequence. Depending on the disorder being treated, combinations of effector genes may be preferred. Examples of suitable combinations are set forth below.

Therapy of Tumors

identical or different, cytostatic, apoptotic, cytotoxic or inflammatory proteins.

identical or different enzymes for the cleavage of the precursors of a cytostatic.

Therapy of Autoimmune Diseases

different cytokines or receptors having synergistic action for the inhibition of the cellular and/or humoral immune reaction.

different or identical TIMPs.

Therapy of Defective Formation of Blood Cells

different, hierarchically consecutive cytokines, such as, for example, IL-1, IL-3, IL-6 or GM-CSF and erythropoietin, G-CSF or thrombopoietin.

Therapy of Nerve Cell Damage

a neuronal growth factor and a cytokine or the inhibitor of a cytokine.

Therapy of Disorders of the Blood Clotting and Blood Circulation System

an antithrombotic and a fibrinolytic (e.g. tPA or uPA).

a cytostatic, apoptotic or cytotoxic protein and an antithrombotic or a fibrinolytic.

a number of different blood clotting factors having a synergistic action, for example Factor VIII and vonwillebrand Factor or Factor VIII and Factor IX.

Vaccinations

an antigen and an immunostimulating cytokine, such as, for example, IL-1α, IL-1β, IL-2, GM-CSF, IL-3 or IL-4 receptor.

different antigens of an infective agent or different infective agents.

different antigens of a tumor type or different tumor types.

Therapy of Viral Infectious Diseases

an antiviral protein and a cytostatic, apoptotic or cytotoxic protein.

antibodies against different surface antigens of a virus or a number of viruses.

Therapy of Bacterial Infectious Diseases

antibodies against different surface antigens and/or toxins of a microorganism.

The insertion of signal sequences and transmembrane domains has been described in Patent Applications DE19639103.2 and DE19651443.6, which are incorporated herein by reference in their entirety. Exemplary techniques are outlined below.

For enhancing translation, the nucleotide sequence GCCACC or GCCGCC can be inserted at the 3′ end of the promoter sequence and directly at the 5′ end of the start signal (ATG) of the signal or transmembrane sequence. Kozak, J. Cell Biol. 108: 299 (1989).

For facilitating the secretion of the expression product of the effector gene, the homologous signal sequence optionally contained in the DNA sequence of the structural gene can be replaced by a heterologous signal sequence improving intracellular secretion. Thus, for example, the signal sequence for immunoglobulin (DNA position≦63 to ≧107; Riechmann et al., Nature 332: 323 (1988)) or the signal sequence for CEA (DNA position≦33 to ≧134; Schrewe et al., Mol. Cell Biol. 10: 2738 (1990); Berling et al., Cancer Res. 50: 6534 (1990)) or the signal sequence of the human respiratory syncytial virus glycoprotein (cDNA of the amino acids≦38 to ≧50 or 48 to 65; Lichtenstein et al., J. Gen. Virol. 77: 109 (1996)) can be inserted.

For anchoring the active protein in the cell membrane of the transfected or transduced cell forming the active protein, a sequence for a transmembrane domain can be introduced alternatively or additionally to the signal sequence. Thus, for example, the transmembrane sequence of the human macrophage colony-stimulating factor (DNA position≦1485 to ≧1554; Cosman et al., Behring Inst. Mitt. 83: 15 (1988)) or the DNA sequence for the signal and transmembrane region of the human respiratory syncytial virus (RSV) glycoprotein G (amino acids 1 to 63 or their part sequences, amino acids 38 to 63; Vijaya et al., Mol. Cell Biol. 8: 1709 (1988); Lichtenstein et al., J. Gen. Virol. 77: 109 (1996)) or the DNA sequence for the signal and transmembrane region of the influenza virus neuraminidase (amino acids 7 to 35 or the subsequence amino acids 7 to 27; Brown et al., J. Virol. 62: 3824 (1988)) can be inserted between the promoter sequence and the sequence of the structural gene.

For anchoring the active protein in the cell membrane of the transfected or transduced cells forming the active protein, the nucleotide sequence for a glycophospholipid anchor can also be inserted. The insertion of a glycophospholipid anchor takes place at the 3′ end of the nucleotide sequence for the structural gene and can additionally take place for the insertion of a signal sequence. Glycophospholipid anchors are described, for example, for CEA, for N-CAM and for further membrane proteins, such as, for example, Thy-1. See Ferguson et al., Ann. Rev. Biochem. 57: 285 (1988).

A further possibility of anchoring active protein to the cell membrane according to the present invention comprises the use of a DNA sequence for a ligand-active compound fusion protein, where the specificity of the ligand of this fusion protein is directed against a membrane structure on the cell membrane of the selected cell. Ligands which bind to the surface of cells include, for example, antibodies or antibody fragments directed against structures on the surface of, for example, endothelial cells, including antibodies against the VEGF receptors or against kinin receptors; muscle cells, such as antibodies against actin or antibodies against angiotensin II receptors or antibodies against receptors for growth factors, such as, for example, against EGF receptors or against PDGF receptors or against FGF receptors or antibodies against endothelin A receptors.

Suitable ligands also include antibodies or their fragments which are directed against tumor-specific or tumor-associated antigens on the tumor cell membrane. Antibodies of this type are described above.

Murine monoclonal antibodies can be humanized. Fab and recombinant Fv fragments and their fusion products can be prepared as described above using technology known in the art.

Further suitable ligands include all active compounds which bind to membrane structures or membrane receptors on the selected cells, such as, for example, cytokines or adhesion molecules, growth factors or their fragments or partial sequences of them, mediators or peptide hormones. For example, these include ligands for endothelial cells, such as IL-1, PDGF, bFGF, VEGF, TGGβ (Pusztain et al., J. Pathol. 169: 191 (1993)) or kinin and derivatives or analogs of kinin; adhesion molecules, such as, for example, SLex, LFA-1, MAC-1, LeCAM-1, VLA-4 or vitronectin and derivatives or analogs of vitronectin (Augustin-Voss et al., J. Cell Biol. 119: 483 (1992); Pauli et al., Cancer Metast. Rev. 9: 175 (1990); Honn et al., Cancer Metast. Rev. 11: 353 (1992); Varner et al., Cell Adh. Commun. 3: 367 (1995)).

The invention is explained in greater detail in the following examples, which are illustrative only and not restrictive.

V. Preparation and Use of the Nucleic Acid Construct

The nucleic acid constructs preferentially consist of DNA. The term nucleic acid constructs is understood as meaning artificial structures of nucleic acid which can be transcribed in the target cells. They are preferably inserted in a vector, plasmid vectors or plasmids complexed with nonviral carriers (Fritz et al., Hum. Gene Ther. 7: 1395 (1996); Solodin et al., Biochem. 34: 13537 (1995); Abdallak et al., Hum Gene Ther. 7: 1947 (1996); Ledley, Hum. Gene Ther. 6: 1129 (1995); Schofield et al., Br. Med. Bull. 51: 56 (1995); Behr, Bioconj. Chem. 5: 382 (1994); Cotten et al., Curr. Opin. Biotechnol. 4: 705 (1993); Hodgson et al., Nature Biotechnol. 14: 339 (1996)) are particularly preferred. The vectors are introduced into the precursor cell of endothelial cells or into endothelial cells using technologies known to those skilled in the art. Cotten et al., Curr. Opin. Biotechnol. 4: 705 (1993); Scheffield et al., Br. Med. Bull. 51: 56 (1995); Ledley, Hum. Gene Ther. 6: 1129 (1995). In a further embodiment, the nucleic acid constructs according to the invention are inserted in a viral vector (Weir et al., Hum. Gene Ther. 7: 1331 (1996); Flotte et al., Gene Ther. 2: 357 (1995); Efstathion et al., Br. Med. Bull. 51: 45 (1995); Kremer et al., Br. Med. Bull. 51: 31 (1995); Vile et al., Br. Med. Bull. 51: 12 (1995); Randrianarison et al., Biologicals 23: 145 (1995); Jolly Cancer Gene Ther. 1: 51 (1994)) and transfected with these endothelial cells. The cells transduced by these means are administered to patients externally or internally, locally, in a body cavity, in an organ, in the blood circulation, in the airway, in the gastrointestinal tract, in the urogenital tract, in a wound cavity or intramuscularly or subcutaneously.

By means of the nucleic acid constructs according to the invention, a structural gene can be expressed cell-specifically and optionally also virus-specifically, under certain metabolic conditions and/or cell cycle-specifically and/or induced by a pharmaceutical, in the endothelial cells or precursor cells of endothelial cells, the structural gene preferably being a gene which codes for a pharmacologically active protein or else for an enzyme which cleaves an inactive precursor of a drug into an active drug. The structural gene can be selected such that the pharmacologically active compound or the enzyme is expressed as a fusion protein with a ligand and this ligand binds to the surface of cells, e.g. proliferating endothelial or tumor cells.

### EXAMPLES

### Example 1

Culturing Endothelial Cells from CD34-positive Blood Cells Without the Use of Fibronectin and Bovine Brain

CD34-positive blood cells, isolated as described by Asahara et al., Science 275: 964 (1997), were cultured either

(a) in plastic bottles, coated with fibronectin and with addition of bovine brain extract (100 μg/ml), as described by Asahara; or

(b) in plastic bottles without fibronectin coating and without addition of bovine brain extract, but with VEGF and bFGF addition (Sigma, in each case 1% v/v), according to this invention.

In each case, the culture medium was medium 199 (Sigma No. M5017) and also included fetal calf serum (FCS, 20%), and the culture medium was incubated at 37° C. and with 5% CO₂ aeration.

After 6 days, the proportion of adherently growing cells forming fusiform and capillary-like structures was determined microscopically and the proportion of endothelial cells was determined by labeling with endothelial cell-specific antibodies (anti CD31, anti vWF, anti Flk 1) with the aid of FACS analysis. No difference in the number and morphology of these cells was found between batch (a) and batch (b). In experimental series of both batches, the proportion of endothelial cells varied between 1 and 10%, which confirms that the addition of the growth factors VEGF and bFGF can replace the coating of the culture bottle with fibronectin and the addition of brain extract.

### Example 2

Culturing Endothelial Cells from Mononuclear Blood Cells

Mononuclear cells were isolated from 120 ml of blood with the aid of centrifugation via a Ficoll gradient and the nonadherent mononuclear blood cells were separated off by incubation for 1 hour in the cell culture bottle and subsequent decantation.

These cells were inoculated into culture bottles according to batch (b) above and cultured for 6 days at 37° C. and 5% strength CO₂ aeration. After 6 days, the proportion of endothelial cells was determined as described under Example 1. It was between 2 and 20% in different experimental series.

### Example 3

Culturing Endothelial Cells from CD14-positive Blood Cells

Mononuclear cells were isolated from 120 ml of blood from a healthy donor with the aid of centrifugation on a Ficoll gradient (Ficoll-Paque, Pharmacia, Uppsala) and the nonadherent mononuclear blood cells were separated off by incubation for 60 min in the cell culture bottle and subsequent decantation. The nonadherent mononuclear cells (NMC) isolated in this way contain 0.3-0.05% of CD34-positive and 5-10% of CD14-positive cells (monocytes and monocyte-like cells).

1×10⁶ NMC were adjusted to 1×10⁶ cells/ml of medium 199 comprising 20% fetal calf serum (both from Gibco) and 100 μg of ECGF (Harbor Bioproducts, Norwood, Mass.) or VEGF (Pepro Techn., London, England) and incubated at 37° C. for 1-3 hours on fibronectin (Harbor Bioproducts, Norwood, Mass.)-coated plastic containers. As a result of this incubation, the content of CD14-positive cells increased from 5-10% to 25-30%.

The NMC was separated off by careful washing and the CD14-positive or CD11-positive cells were isolated using magnetic beads, coated with anti-CD14 or anti-CD11 (CD14/CD11 Micro Beads, Miltenyi Biotec, Bergisch-Gladbach, Germany) according to the instructions of the manufacturer.

NMC comprising about ≧80% CD14-positive cells were incubated in the above-mentioned medium 199, supplemented with FCS and ECGF or VEGF, in fibronectin-coated plastic containers at 37° C. with 5% CO₂ in a humidified atmosphere.

The cells in the culture were investigated with the aid of monoclonal antibodies and with the aid of RT-PCR after 6 hours, 3 days and 5 days.

After 6 hours, small mononuclear CD14-positive cells were already observed, which were positive for the endothelial cell-specific markers acetyl LDL receptors, CD34, Flk-1 and von Willebrand Factor. On the 3rd day, these cells showed strong signs of proliferation. On day 5, adherent large granular oval cells and spindle cells were observed, which all carried the endothelial cell markers mentioned, but no longer the CD14 marker.

As soon as these endothelial cells became confluent, they additionally expressed VE-cadherin. After 1 to 2 weeks, >80% of the cells in the culture were endothelial cells.

### Example 4

Endothelial Cell-specific Transformation and Culture of Endothelial Cells from Mononuclear Blood Cells

Mononuclear cells are isolated from 120 ml of blood with the aid of centrifugation on a Ficoll gradient and the nonadherent mononuclear blood cells are separated off by incubation for 1 hour in the cell culture bottle and subsequent decantation. These blood cells are adjusted to a concentration of 1×10⁷/ml of culture medium, inoculated into 60 mm culture dishes and incubated at 37° C. for 10 min with a complex of the plasmid according to the invention and Superfect (Quiagen).

The preparation of the complex is carried out according to the instructions of the manufacturer of Superfect.

A plasmid containing the following DNA sequences in the reading frame from 5′ to 3′ is used:

the promoter of the human endoglin gene (NS1-2415; Patent Application D19704301.1)

the cDNA of the human cyclin-dependent kinase 4 (cdk-4) having a mutation in the codon 24 (replacement of an arginine (CGT) by a cysteine (TGT), Wölfel et al. Science 269: 1281 (1997)).

the nuclear localization signal (NLS) of SV40 (SV40 large T; amino acids 126 to 132; PKKKRKV (SEQ ID NO:3); Dingwall et al., TIBS 16: 478 (1991)).

Linkage of the individual constituents of the construct is carried out by means of suitable restriction sites which are carried along by means of PCR amplification to the termini of the various elements. The linkage is carried out with the aid of enzymes and DNA ligases specific for the restriction sites, which are known to those skilled in the art. These enzymes are commercially available. The nucleotide prepared in this way is cloned in with the aid of these enzymes.

After incubation of the mononuclear blood cells with the Superfect/plasmid complex, the blood cells are washed and cultured in cell culture medium as described in Example 2. After 6 days, the proportion of endothelial cells is determined as described in Example 1. In different experimental series, it varies between 10 and 60%.

### Example 5

Preparation and Use of Transduced Endothelial Cells as Vectors

Endothelial cells, isolated, transduced and proliferated as described in Example 4, are inoculated into 60 mm culture dishes and incubated at 37° C. for 10 min with a complex of a further plasmid according to the invention and Superfect (Quiagen). The preparation of this complex is carried out according to the instructions of the manufacturer of Superfect.

The plasmid according to the invention contains the following DNA sequences in the reading frame from 5′ to 3′:

Activator Component A

the promoter of the cdc25C gene (nucleic acids 290 to +121; Zwicker et al., EMBO J. 14, 4514 (1995);

Zwicker et al., Nucl. Acids Res. 23, 3822 (1995));

the nuclear localization signal (NLS) of SV40 (SV40 Large T, amino acids 126-132; PKKKRKV (SEQ ID NO.: 3), Dingwall et al., TIBS 16, 478 (1991));

the acidic transactivation domain (TAD) of HSV-1 VP16 (amino acids 406 to 488; Triezenberg et al.,

Genes Developm. 2, 718 (1988); Triezenberg, Curr. Opin. Gen. Developm. 5, 190 (1995));

the cDNA for the cytoplasmatic part of the CD4 glycoprotein (amino acids 397-435; Simpson et al., Oncogene 4, 1141 (1989); Maddon et al., Cell 42, 93 (1985))

Activator Subunit B:

the promoter of the human endoglin gene (nucleic acids 1 to 2415; Patent Application D19704301.1);

the nuclear localization signal (NLS) of SV40 (SV40 large T; amino acids 126-132 PKKKRKV (SEQ ID NO:3);

Dingwall et al., TIBS 16, 478 (1991));

the cDNA for the DNA-binding domain of the Gal4 protein (amino acids 1 to 147, Chasman and Kornberg, Mol. Cell. Biol. 10, 2916 (1990));

the cDNA for the CD4 binding sequence of the p56 lck protein (amino acids 1-71; Shaw et al., Cell 59, 627 (1989); Turner et al., Cell 60, 755 (1990);

Perlmutter et al., J. Cell. Biochem. 38, 117 (1988))

Activator-responsive Promoters:

10× the binding sequence for Gal4 binding protein having the nucleotide sequence 5′-CGGACAATGTTGACCG-31 (SEQ ID NO:4, Chasman and Kornberg, Mol. Cell. Biol. 10, 2916 (1989));

the basal promoter of SV40 (nucleic acids 48 to 5191; Tooze (ed). DNA Tumor Viruses (Cold Spring Harbor New York, N.Y., Cold Spring Harbor Laboratory);

Effector Gene:

the cDNA for the human β-glucuronidase (nucleotide sequence 93 to 1982; Oshima et al., PNAS USA 84, 65 (1987)).

The functioning of the described activator sequence is as follows. The promoter cdc25B regulates cell cycle-specifically the transcription of the combined cDNAs for the activation domain of VP16 and the cytoplasmatic part of CD4 (activation subunit A). The promoter of the human endoglin gene regulates endothelial cell-specifically the transcription of the combined cDNAs for the DNA binding protein of Gal4 and the CD4-binding part of the p56 lck protein (activation subunit B).

The expression products of the activator subunits A and B dimerize by the binding of the CD4 domain to the p56 lck domain. The dimeric protein is a chimeric transcription factor for the activator-responsive promoter (DNA sequence for the Gal4 binding domains/the SV40 promoter) for the transcription of the effector gene (luciferase gene).

The linkage of the individual constituents of the construct is carried out by means of suitable restriction sites which are carried along by means of PCR amplification to the termini of the various elements. The linkage is carried out with the aid of enzymes and DNA ligases specific for the restriction sites, which are known to those skilled in the art. These enzymes are commercially available.

With the aid of these enzymes, the nucleotide construct prepared in this way is cloned into the pXP2 plasmid vector (Nordeen, BioTechniques 6, 454 (1988)). After incubation of the mononuclear blood cells with the Superfect/plasmid complex, the blood cells are washed and cultured in cell culture medium as described in Example 2.

After 6 days, the amount of β-glucuronidase produced by the endothelial cells is measured with the aid of 4-methylumbelliferyl-β-glucuronide as substrate.

To check the cell cycle specificity, endothelial cells are synchronized in G₀/G₁ over 48 hours by withdrawal of methionine. The DNA content of the cells is determined in a fluorescence activation cell sorter after staining with Hoechst 33258 (Lucibello et al., EMBO J. 14, 132 (1995)).

The following results are obtained:

In non-transfected endothelial cells, no increase in β-glucuronidase in comparison with nontransected fibroblasts can be determined. Transfected endothelial cells express markedly more β-glucuronidase than nontransfected endothelial cells. Proliferating endothelial cells (DNA >2S; S=simple chromosome sets) secrete markedly more β-glucuronidase than in G₀/G₁ synchronized endothelial cells (DNA=2S).

This demonstrates that the activator-responsive promoter unit described above leads to a cell-specific, cell cycle-dependent expression of the structural gene for β-glucuronidase.

### Example 6

Gene Therapy with the Transformed Cells of the Invention

Endothelial cells transformed with effector genes as described above are administered to a patient in need of gene therapy of a disorder. The administration is effected locally. The endothelial cells preferably populate regions with cell damage, and due, to the cell cycle- and endothelial cell-specificity of the activator-responsive promoter unit mainly, if not exclusively, proliferating endothelial cells secrete β-glucuronidase. This β-glucuronidase cleaves a subsequently injected, highly tolerable, doxorubicin, inhibiting endothelial cell proliferation, and acts cytostatically on these cells and on adjacent tumor cells. As a result, tumor growth is inhibited.

Priority applications DE 19731154.7, filed Jul. 21, 1997, and DE 19752299.8, filed Nov. 26, 1997, are incorporated herein by reference in their entirety, including their specifications, claims, and drawings. All references cited herein, including patents, patent applications, articles and books, also are incorporated by reference herein in their entirety.

What is claimed is:

1. A method of culturing mononuclear cells, comprising:
isolating mononuclear cells from the blood or cell-containing fluids of the body of a mammal;
culturing the cells in a culture medium comprising one or more of gangliosides, phospholipids, glycolipids and growth factors for endothelial cells.

2. The method of claim 1, wherein the cells are selected from the group consisting of CD34-, CD14-, CD11-, CD11b-, CD13-, CD64- and CD68-positive cells and endothelial cells.

3. The method of claim 1, wherein the cells are isolated from blood in veins, capillaries, arteries, umbilical cord or placenta, or from bone marrow, spleen, lymph nodes, peritoneal space, pleural space, lymph, veins, arteries, or capillaries, or from connective tissue fluid.

4. The method of claim 1, wherein the culture medium comprises one or more growth factors for endothelial cells.

5. The method of claim 4, wherein the growth factors are selected from the group consisting of growth factors influencing differentiation, survival, migration and vascularization.

6. The method of claim 1, wherein the growth factors are selected from the group consisting of ECGF, FGFα, FGFβ, ECAF, IGF-1; IGF-2; Sl-3; EGF; SCF, TGFβ, Tie-2-ligands, stromal derived Factor-1, GM-CSF, G-CSF, M-CSF, Sl-4, Sl-1, CSF-1, Sl-8, PDGF, TFNα, oncostatin M, BG1, platelet derived endothelial cell growth factor, TNFα, angiogenin, pleiotrophin, VEGF, VEGF-B, VEGF-C, VEGF-D, neuropilin, and Flt-3 ligand.

7. A method of making cells capable of expressing a biologically active protein, comprising:
isolating mononuclear cells from the blood or cell-containing fluids of the body of a mammal;
culturing the cells in a culture medium comprising one or more of gangliosides, phospholipids, glycolipids and growth factors for endothelial cells;
optionally, before or after culturing the cells, immortalizing the cells; and
transfecting the cells with a nucleic acid construct comprising a gene for the biologically active protein.

8. The method of claim 7, wherein the cells are selected from the group consisting of CD34-, CD14-, CD11-, CD11b-, CD13-, CD64- and CD68-positive cells and endothelial cells.

9. The method of claim 7, wherein the cells are isolated from blood in veins, capillaries, arteries, umbilical cord or placenta, or from bone marrow, spleen, lymph nodes, peritoneal space, pleural space, lymph, veins, arteries, or capillaries, or from connective tissue fluid.

10. The method of claim 7, wherein the culture medium comprises one or more growth factors for endothelial cells

11. The method of claim 10, wherein the growth factors are selected from the group consisting of growth factors influencing differentiation, survival, migration and vascularization.

12. The method of claim 7, wherein the cells are immortalized by a process selected from the group consisting of transforming the cells with an exogenous oncogene; activating an endogenous oncogene; and inactivating an endogenous suppressor gene.

13. The method of claim 12, wherein the exogenous oncogene is linked to an endothelial cell-specific promoter which controls transcription of the oncogene.

14. The method of claim 12, wherein the endogenous suppressor gene is inactivated upon expression of a nucleotide sequence linked to an endothelial cell specific promoter which controls transcription of the nucleotide sequence.

15. The method of claim 7, wherein the biologically active protein is selected from the group consisting of cytokines, chemokines, and growth factors; receptors for cytokines, chemokines and growth factors; proteins having antiproliferative or cytostatic or apoptotic action; antibodies; antibody fragments; angiogenesis inhibitors; peptide hormones; clotting factors; clotting inhibitors; fibrinolytic proteins, peptides or proteins acting on the blood circulation; blood plasma proteins; and antigens of infective agents, cells or tumors, wherein the antigens trigger an immune response.

16. The method of claim 7, wherein the biologically active protein is an enzyme which cleaves a precursor of a drug into a drug.

17. The method of claim 7, wherein the biologically active protein is a ligand-active compound fusion protein or a ligand-enzyme fusion protein, wherein the ligand is selected from the group consisting of cytokines, growth factors, antibodies, antibody fragments, peptide hormones, mediators, cell adhesion proteins and LDL receptor-binding proteins.

18. The method of claim 7, wherein the nucleic acid construct comprises a promoter operably linked to the gene for the biologically active protein.

19. The method of claim 18, wherein the promoter operates cell-specifically, cell cycle specifically, virus specifically, metabolically or by hypoxia.

20. The method of claim 18, wherein the promoter is inducible.

21. The method of claim 7, wherein the nucleic acid construct comprises at least two promoters operably linked to the gene for the biologically active protein, which promoters may be the same or different.

22. A method of effecting gene therapy of a disorder, comprising:
administering to a patient in need thereof a therapeutically effective amount of cells obtained by
isolating mononuclear cells from the blood or cell-containing fluids of the body of a mammal;
culturing the cells in a culture medium comprising one or more of gangliosides, phospholipids, glycolipids and growth factors for endothelial cells;
optionally, before or after culturing the cells, immortalizing the cells; and
transfecting the cells with a nucleic acid construct comprising a gene for a biologically active protein useful in the prophylaxis or therapy of the disorder.

23. The method of claim 22, wherein the biologically active protein is selected from the group consisting of cytokines, chemokines, and growth factors; receptors for cytokines, chemokines and growth factors; proteins having antiproliferative or cytostatic or apoptotic action; antibodies; antibody fragments; angiogenesis inhibitors; peptide hormones; clotting factors; clotting inhibitors; fibrinolytic proteins, peptides or proteins acting on the blood circulation; blood plasma proteins; and antigens of infective agents, cells or tumors, wherein the antigens trigger an immune response.

24. The method of claim 22, wherein the biologically active protein is an enzyme which cleaves a precursor of a drug into a drug.

25. The method of claim 22, wherein the biologically active protein is a ligand-active compound fusion protein or a ligand-enzyme fusion protein, wherein the ligand is selected from the group consisting of cytokines, growth factors, antibodies, antibody fragments, peptide hormones, mediators, cell adhesion proteins and LDL receptor-binding proteins.

26. The method of claim 22, wherein the cells are administered externally, orally, intravesically, nasally, intrabronchially, subcutaneously, into the gastrointestinal tract, or are injected into an organ, into a body cavity, into musculature, or into blood circulation.

27. The method of claim 22, wherein the disorder is selected from the group consisting of tumors, leukemias, autoimmune disorders, allergies, arthritides, inflammations, organ rejections, transplant-host reactions, blood clotting disorders, circulation disorders, anemia, infections, hormone disorders and central nervous system (CNS) damage.

28. A method of endothelializing injured vessels, comprising,
administering to a patient in need thereof a therapeutically effective amount of cells obtained by
isolating mononuclear cells from the blood or cell-containing fluids of the body of a mammal;
culturing the cells in a culture medium comprising one or more of gangliosides, phospholipids, glycolipids and growth factors for endothelial cells; and
optionally, before or after culturing the cells, immortalizing the cells.

29. A cell obtainable by:
(a) isolating mononuclear cells from the blood or cell-containing fluids of the body of a mammal; and
(b) culturing the mononuclear cells in a cell culture medium comprising one or more of gangliosides, phospholipids, glycolipids and growth factors for endothelial cells.

30. A pharmaceutical composition comprising the cell of claim 29 and a pharmaceutically acceptable vehicle, carrier, or diluent.

31. A cell for use in gene therapy, obtainable by
(a) isolating mononuclear cells from the blood or cell-containing fluids of the body of a mammal;
(b) culturing the mononuclear cells in a cell culture medium comprising one or more of gangliosides, phospholipids, glycolipids and growth factors for endothelial cells;
(c) optionally, before or after culturing the cells, immortalizing the cells by a process selected from the group consisting of transforming the cells with an exogenous oncogene; activating an endogenous oncogene; and inactivating an endogenous suppressor gene; and
(d) transfecting the cells with a nucleic acid construct comprising a gene coding for a biologically active protein, wherein the nucleic acid construct optionally comprises one or more promoters for expressing the gene for the biologically active protein cell-specifically, cell cycle-specifically, virus-specifically, metabolically or by hypoxia.

32. A pharmaceutical composition comprising the cell of claim 31 and a pharmaceutically acceptable vehicle, carrier, or diluent.
